(19) United States
(12) Patent Application Publication
(10) Pub. No.: US 2006/0013880 A1
(43) Pub. Date: Jan. 19, 2006

(54) Formulation stabilizer for proton pump inhibitors

(76) Inventors: Robert Femia, Kinnelon, NJ (US); Narayan Ragunathan, West Nyack, NY (US); Joseph Zhou, Denville, NJ (US); Ping He, Garnerville, NY (US)

Correspondence Address: Supervisor, Patent Prosecution Services;PIPER RUDNICK LLP, 1200 Nineteenth Street, N.W., Washington, DC, 20036-2412, US

(21) Appl. No.: 10/893,489

(22) Filed: Jul. 19, 2004

(51) Int. Cl. A61K 31/4439; A61K 9/14

(52) U.S. Cl. 424/486; 514/338

(57) ABSTRACT

The present invention provides a composition containing a benzimidazole derivative proton pump inhibitor and a polymeric base. The polymeric base is cholestyramine-OH, Eudragit E-PO, chitosan, or a mixture thereof. The composition stabilizes the benzimidazole derivative proton pump inhibitor in a humid environment so that degradation during storage.

### BACKGROUND OF THE INVENTION

1. Field of the Invention

The present invention relates to pharmaceutical formulations and, in particular, pharmaceutical formulations that stabilize benzimidazole derivative proton pump inhibitors.

2. Background of the Technology

Proton pump inhibitors, such as omeprazole, lansoprazole, pantoprazole, leminoprazole, pariprazole, rabeprazole, esomeprazole, and other benzimidazole derivatives, are used as anti-ulcer drugs to inhibit gastric acid secretion. The benzimidazole derivatives, however, are susceptible to degradation/transformation in acidic and neutral media and require special formulations to provide suitable pharmaceutical dosage form. Since the degradation is catalyzed by acidic reacting compounds, benzimidazole derivative proton pump inhibitors in an oral solid dosage form must be protected from contact with the acidic gastric fluid. The active drug substance must be transferred in intact form to the intestine where pH is less acidic, neutral or alkaline and where rapid absorption of the proton pump inhibitors can occur. Moreover, benzimidazole derivative proton pump inhibitors degrade rapidly in humid conditions, even at ambient humidity and temperature, leading to the loss of their bioactivity during storage. In order to minimize degradation during storage and degradation in the stomach, it is necessary to formulate the benzimidazole derivative proton pump inhibitors so that their activity can be maintained.

The pharmaceutical dosage forms of benzimidazole derivative proton pump inhibitors can be protected from contact with acidic gastric fluid by an enteric coating layer. Enteric coating is by far the most popular method of protecting an acid-labile drug from gastric degradation. In this method, the dosage form is coated with a polymer that does not dissolve in the low pH gastric environment, but dissolves in the alkaline environment of the small intestine. Ordinary enteric coating layers, however, comprise compounds which contain acidic groups. If covered with such an enteric coating layer, the acid labile benzimidazole derivatives may rapidly decompose by direct or indirect contact with the acidic groups of the coating layer.

Various stabilizing agents for benzimidazole derivative proton pump inhibitors have been developed ( see, for example, U.S. Pat. Nos. 4,628,098; 4,853,230; 4,026,560; 5,689,333; 5,045,321; 5,093,132; 5,433,959; and 6,013,281). It was found that benzimidazole derivative proton pump inhibitors are stabilized in the presence of basic inorganic salts of magnesium, calcium, potassium and sodium. The stability can be further consolidated by separating the acidic components of the enteric coat by an intermediate coating, where the core material are pellets.

In the method described above, however, a large quantity of the stabilizing inorganic salt, such as sodium carbonate, must be administered with each dose of proton pump inhibitors. There is a major disadvantage in using large quantities of sodium bicarbonate orally, since sodium bicarbonate, upon neutralization in the gastric fluid, produces gases, causing flatulence and belching (see e.g. U.S. Pat. No. 5,840,737), which is detrimental to patients suffering from gastro-esophageal reflux disease.

European Pat. Appl. No. 0998944 describes pharmaceutical formulations reflecting enteric coated formulation that eliminates the separating layer. The formulation comprises a core and an enteric coating layer. The core contains a non-covalent complex of the benzimidazole derivative, such as lansoprazole, and an anion exchange resin, such as cholestyramine chloride or Dowex. The enteric coating is intended to protect against exposure of the benzimidazole derivative due to gastric juices. To avoid undesirable reactions with the benzimidazole/stabilizer core, the acid substitution on the enteric coated is limited. This reference generally teaches the selection of cholestyramine as a stabilizer. It does not distinguish between variant forms, but refers to Duolite® AP-143. This pharmaceutical grade resin is cholestyramine chloride. The inventors have discovered that cholestyramine of this character does not provide significant stabilization of benzimidazole proton pump inhibitors such as lansoprazole and omeprazole under conventional storage conditions for which instability is aggravated under conditions of greater humidity. Accordingly, there still exist a need for a pharmaceutical composition that is well tolerated by patients and stable against degradation during storage and upon ingestion, and can be easily manufactured.

### SUMMARY OF THE INVENTION

The present invention provides a composition containing a benzimidazole derivative proton pump inhibitor and a polymeric base selected from the group consisting of cholestyramine-OH, Eudragit E-PO, chitosan, or a mixture thereof. The composition of the present invention provides superior stability for the benzimidazole derivative proton pump inhibitor under naturally occurring humidity ranges so that degradation during dosage storage and in the stomach is minimized. Moreover, the composition of the present invention can be easily manufactured by directly admixing the benzimidazole derivative proton pump inhibitor with the polymeric base.

In a preferred embodiment, the benzimidazole derivative proton pump inhibitor is one of lansoprazole, pantoprazole, leminoprazole, pariprazole, omeprazole, rabeprazole, and esomeprazole. In a more preferred embodiment, the benzimidazole derivative proton pump inhibitor is lansoprazole.

In another embodiment, the benzimidazole derivative proton pump inhibitor and the polymeric base are both in powder form.

In yet another embodiment, the benzimidazole derivative proton pump inhibitor and polymeric base have a benzimidazole derivative/polymeric base weight ratio of between 0.1 and 0.9, and preferably between 0.3 to 0.6.

In yet another embodiment, the pharmaceutical composition further comprises at least one pharmaceutically acceptable excipient.

In yet another embodiment, the composition is formulated in a dosage form for oral administration. For oral administration, a conventional enteric coating is preferably employed, and preferably pharmaceutical grade benzimidazole derivative proton pump inhibitors and polymeric bases are utilized.

Another aspect of the invention relates to a method for stabilizing a benzimidazole derivative proton pump inhibitor in a pharmaceutical composition. The method comprises the step of admixing a benzimidazole derivative proton pump inhibitor with a polymeric base. The polymeric base is cholestyramine-OH, Eudragit E-PO, chitosan, or a mixture thereof.

Yet another aspect of the present invention relates to a method for prophylaxis and treatment of gastric acid disorders. The method comprises the step of administering to a patient in need of such treatment a therapeutically effective amount of the pharmaceutical composition of the present invention.

### DETAILED DESCRIPTION OF THE INVENTION

One aspect of the present invention provides a pharmaceutical composition that stabilizes benzimidazole derivative proton pump inhibitors in humid environment. The term “humid environment” refers to naturally occurring humidity levels at temperatures ordinarily experienced in the developed world. Thus, at a given temperature, an atmospheric humidity range of about 20% to 100% or a free water content (not including molecularly bound water such as the water molecules in CaCl₂.2H₂O) of 5% and greater in the pharmaceutical composition is considered as a “humid environment.” The composition contains a mixture of at least one benzimidazole derivative proton pump inhibitor and a polymeric base selected from the group consisting of cholestyramine hydroxide, Eudragit E-PO, and chitosan. The weight ratio between the benzimidazole derivative proton pump inhibitor and the polymeric base may vary depending on a particular benzimidazole derivative/polymeric base combination. Preferably, the benzimidazole derivative/polymeric base weight ratio is between 0.1 and 0.9. More preferably, the benzimidazole derivative/polymeric base weight ratio is between 0.2 and 0.8. Preferably, both the benzimidazole derivative proton pump inhibitor and the polymeric base are in powder form.

Cholestyramine is a strongly basic anion exchange resin consisting of a copolymer of styrene and divinylbenzene with quaternary ammonium functional groups. The functional group of the cholestyramine resin is shown in Formula I.

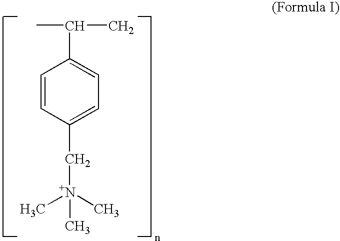

Cholestyramine is quite hydrophilic, but is insoluble in water and is not absorbed from the digestive tract. It has been used as an excipient in pharmaceutical preparations. Cholestyramine is also a bile acid sequestrant and has been used as an active drug ingredient (e.g. Cholestyramine Resin USP) to lower cholesterol levels. Cholestyramine resin is commercially available in chloride form (cholestyramine chloride or cholestyramine-Cl), which can be converted to cholestyramine hydroxide (cholestyramine-OH) by reacting with a strong base, such as NaOH. As demonstrated in the Examples, cholestyramine chloride fail to stabilize benzimidazole derivatives under humid conditions. However, cholestyramine hydroxide provides a surprising stabilizing effect to benzimidazole derivatives under humid conditions.

Preferably, the cholestyramine-OH resin is in a powder form and at least 80% of the cholestyramine-OH particles have a diameter of 500 micron or less. More preferably, the cholestyramine-OH resin has an exchange capacity of 0.1-2.4 g sodium glycocholate/g resin and a dry substance content of 80% or more.

Eudragit E-PO is a fine powder made from Eudragit E100, which is a cationic copolymer based on demethylaminoethyl methacrylate and neutral methacrylic esters. The structure of Eudragit E is shown in Formula II.

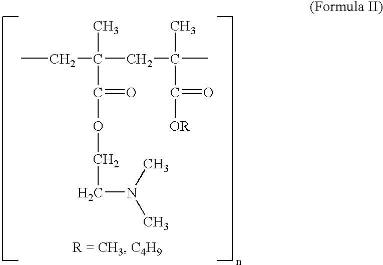

Eudragit E-PO is normally employed as an aqueous emulsion in combination with hydrophobic plasticizers or fatty acids (C12-C18) and ionic emulsifiers to manufacture protective and insulating coatings that dissolve in gastric acid for solid pharmaceutical dosage forms. The coatings are also suitable for improving moisture protection and for masking taste. In addition, Eudragit E-PO can be combined with plasticizers, crosslinkers, active ingredients and further excipients such as permeation enhancers, to form self-adhesive matrix systems for dermal and transdermal applications via aqueous, organic or hotmelt processes. Prior to the present invention, however, Eudragit E-PO has not been used as a stabilizer for proton pump inhibitor, especially in a powder form. It is now unexpectedly found that Eudragit E-PO provides surprising stabilizing effect to benzimidazole derivatives in a humid environment.

Preferably, the Eudragit E-PO is in a powder form and has an average molecular weight of about 150,000. More preferably, the Eudragit E-PO powder has a dry substance content of 95% or more and at least 80% of the Eudragit E-PO particles have a diameter of 400 micro or less. The dimethylaminoethyl (DMAE) group content on dry Eudragit E-PO is preferably between 15-30% by weight.

Chitosan is a natural product derived from chitin, a polysaccharide found in the exoskeleton of insects and shellfish like shrimp or crabs. Chitosan the principal derivative of chitin, produced by alkaline deacetylation of chitin. The structure of Chitosan (poly[β-(1-4)-2-amino-2-2deoxy-D-glucopyranose] is shown in Formula III. The typical commercial chitosan has approximately 85% deacetylation.

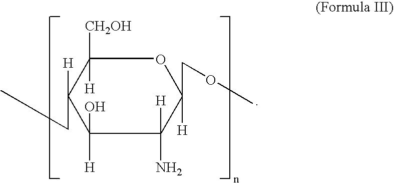

Chitosan is chemically similar to cellulose, which is the major composition of plant fiber, and possesses many properties as fiber. Chitosan has been used as a water swellable material in pharmaceutical compositions. For example, U.S. Pat. Appl. No. 20030133985 describes an erodible, gastric-retentive drug dosage form comprising a pharmacologically active agent incorporated in a matrix of biocompatible, hydrophilic polymer including chitosan. Because of its ability to bind fat in the digestive tract, chitosan has also been used as a weight loss product and a dietary supplement to lower serum cholesterol levels. Prior to the present invention, however, chitosan has not been used as a stabilizer for proton pump inhibitors. It is now unexpectedly found that chitosan provides surprising stabilizing effect to benzimidazole derivatives.

Preferably, a base form of chitosan is used in a powder form and has an average diameter of 2-500 micron. More preferably, the base powder form of chitosan has a dry substance content of at least about 95%.

Examples of the benzimidazole derivative proton pump inhibitors include, but are not limited to, lansoprazole, pantoprazole, leminoprazole, pariprazole, omeprazole, rabeprazole, and esomeprazole. Preferably, the benzimidazole derivative proton pump inhibitor is lansoprazole. More preferably, the lansoprazole is supplied in powder form.

The pharmaceutical composition of the present invention may further contain one or more pharmaceutically acceptable excipients.

Examples of the pharmaceutically acceptable excipients include, but are not limited to, surfactants, plasticizers, fillers, lubricants, preservatives, sweetener agents, flavoring agents, pharmaceutical-grade dyes or pigments, and viscosity agents.

The surfactants can be non-ionic hydrophilic surfactants, ionic hydrophilic surfactants, or hydrophobic surfactants. Examples of the non-ionic hydrophilic surfactant include, but are not limited to, alkylglucosides; alkylmaltosides; alkylthioglucosides; lauryl macrogolglycerides, polyoxyethylene alkyl ethers, polyoxyethylene alkylphenols; polyethylene glycol fatty acids esters, polyethylene glycol glycerol fatty acid esters; polyoxyethylene sorbitan fatty acid esters, polyoxyethylene-polyoxypropylene block copolymers, polyglycerol fatty acid esters, polyoxyethylene glycerides; polyoxyethylene sterols; polyoxyethylene vegetable oils, polyoxyethylene hydrogenated vegetable oils, tocopherol polyethylene glycol succinates, sugar esters, sugar ethers, and sucroglycerides.

Examples of the ionic hydrophilic surfactant include, but are not limited to, alkyl ammonium salts; bile acids and salts, analogues, and derivatives thereof; fatty acid derivatives of amino acids, carnitines, oligopeptides, and polypeptides; glyceride derivatives of amino acids, oligopeptides, and polypeptides; acyl lactylates; mono- and diacetylated tartaric acid esters of mono- and diglycerides; succinylated monoglycerides; citric acid esters of mono- and diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; and sodium docusate.

Examples of the hydrophobic surfactant include, but are not limited to, alcohols; polyoxyethylene alkylethers; fatty acids; bile acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils.

Examples of the plasticizer include, but are not limited to, plasticizers, such as polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate.

Examples of the fillers include, but are not limited to, lactose, sucrose, maltodextrin, and microcrystalline cellulose.

Examples of the lubricants include, but are not limited to, magnesium stearate, stearic acid, and talc.

Examples of the preservatives include, but are not limited to, phenol, alkyl esters of parahydroxylbenzoic acid, benzoic acid and the salts thereof, boric acid and the salts thereof, sorbic acid and the salts thereof, chlorbutanol, benzyl alcohol, thimerosal, phenylmercuric acetate and nitrate, nitromersol, benzalkonium chloride, cetylpyridinium chloride, methyl paraben, and propyl paraben.

Examples of the sweeteners include, but are not limited to, maltose, sucrose, glucose, sorbitol, glycerin and dextrins, and artificial sweeteners, such as aspartame, saccharine and saccharine salts.

The flavoring agents include those described in Remington's Pharmaceutical Sciences, 18^{th} Edition, Mack Publishing Company, 1990, pp. 1288-1300, incorporated by reference herein.

The dyes or pigments include those described in Handbook of Pharmaceutical Excipients, pp. 81-90, 1986 by the American Pharmaceutical Association & the Pharmaceutical Society of Great Britain, incorporated by reference herein.

Examples of the viscosity agents include, but are not limited to, methylcellulose, sodium carboxymethylcellulose, hydroxypropylmethylcellulose, carbomer, povidone, acacia, guar gum, xanthan gum, and tragacanth. Particularly preferred viscosity agents are methylcellulose, carbomer, xanthan gum, guar gum, povidone, and sodium carboxymethylcellulose.

In a preferred embodiment, the pharmaceutical composition of the present invention is a coated with an enteric coating. The enteric coating typically contains to a mixture of pharmaceutically acceptable excipients which is applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. It should be noted that enteric coating may increase the amount of degradation of the active ingredient. The enteric coating may include an acid-resistant material, preferably one that can resists acids up to a pH of above about 5.0 or higher. Exemplary acid-resistant materials include, cellulose acetate phthalate, hydroxypropylmethylcellulose phthalate, polyvinyl acetate phthalate, carboxymethylethylcellulose, Eudragit L or Eudragit S, and mixtures thereof.

The enteric coating agent may also include an inert processing aid in an amount of about 10-80 wt %, and preferably about 30-50 wt %, based on the total weight of the acid-resistant material and the inert processing aid. Exemplary materials suitable for uses as the inert processing aid includes, finely divided forms of talc, silicon dioxide, magnesium stearate etc. The enteric coating may further comprise a moisture-resistant component. Typical solvents which may be used to apply the acid resisting component-inert processing aid mixture include isopropyl alcohol, acetone, methylene chloride and the like. An aqueous suspension of the enteric coating agent can also be used for processing. Generally the acid-resistant material-inert processing aid mixture will comprise about 5-20 wt % of the mixture based on the total weight of the solvent and the mixture. Finally, when an enteric coat is included in the formulation, there may also be at least one layer of seal coating or separation coating between the drug-containing composition and the enteric coat. Such layers are typically made of an inert material such as an acid- and alkaline-resistant material. Additional additives and their levels, and selection of a primary coating material or materials will depend on the resistance to dissolution and disintegration in the stomach; the impermeability to gastric fluids and drug/carrier/enzyme while in the stomach; the ability to dissolve or disintegrate rapidly at the target intestine site; the physical and chemical stability during storage; the non-toxicity; easy application as a coating (substrate friendly); and economical practicality.

Another aspect of the present invention pertains to a method of preparing the benzimidazole derivative/polymeric base mixture. Various techniques and processes may be used to prepare the benzimidazole derivative/polymeric base mixture. In a preferred embodiment, both the benzimidazole derivative and the polymeric base are in powder form. The mixture is prepared by weighing a desired amount of each substance and thoroughly admixing the two dry powders such that the benzimidazole derivative is evenly dispersed among the polymeric base.

Yet another aspect of the present invention relates to a method for prophylaxis or treatment of peptic ulcers. The method contains the step of administering an effective amount of the pharmaceutical composition of the present invention to a subject. A preferred route of administration is oral administration.

Dosages for the pharmaceutical composition should be adjusted depending on the age, weight, and condition of the subject, as well as on the route and dosage form of administration, daily regulations, and the desired results. Dosage forms of the pharmaceutical composition can also be formulated as enteric coated delayed release oral dosage forms, i.e., as an oral dosage form which utilizes an enteric coating to effect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

### EXAMPLES

### Example 1

### Preparation of Cholestyramine Hydroxide (Cholestyramine-OH)

Five grams of cholestyramine chloride (Lot# 038167, Dow Chemical) was suspended in 100 ml of deionized water (DI water). Five grams of NaOH (Fisher Chemicals, Fairlawn, N.J.) was added to the suspension and stirred for 30 minutes. The suspension was filtered through a buckner funnel. The solid resin material was washed with DI water 4 times until pH became neutral, collected and dried at 60° C. for about 16 hours.

### Example 2

### Preparation of Binary Mixture for Standardized Stress Study

This standardized stress study simulates degradation during long-term storage under high, but naturally occurring humidity levels.
1) Dry binary mixture was prepared by accurately weighing 30 mg of lansoprazole ( Lot # A10344, purchased from ITF Chemical, LTDA, of Polo Petrochimico, Brazil) into a 20 ml head space-gas chromatography vial. A stabilizer was also weighed and added into the vial. The stabilizer tested in the experiments included the polymeric bases: cholestyramine chloride ( Lot# 038167, Dow Chemical), cholestyramine resin in hydroxide form (prepared from cholestyramine chloride as described in Example 1), Eudragit E-PO powder (lot# G030131033, Rohm Gmbh, Germany), and chitosan powder (Mutchler inc., Harrington, N.J.). Sodium bicarbonate (Fisher Chemicals, Fairlawn, N.J.) was used as a stabilizer control. The lansoprazole and stabilizer were mixed by vortexing for 30 seconds.
2) A second set of dry binary mixtures were prepared in a head space-gas chromatography vial with a glass insert. The glass insert contains 20 μl of DI water which, under the testing temperature of 60° C., to create a relative humidity of about 100% in the vial.
3) All the vials were sealed by crimping with Telfon lined rubber caps and were placed upright in an oven at 60° C. for 10 days.
4) The stressed sample was transferred into a 100 ml volumetric flask, dissolved and diluted to volume with methanol. The solution was filtered with a 0.45 μm Millex filter.
5) Five milliliters of above filtered solution was diluted to 25 ml with 1% diethanolamine in DI water. Ten microliters of the diluted samples were analyzed by HPLC to quantitate the remaining lansoprazole using freshly prepared external lansoprazole standard solution. The HPLC conditions are disclosed in Example 5.

### Example 3

### Stability of Lansoprazole in Various Lansoprazole-Stabilizer Mixtures

Tables 1 and 2 list the lansoprazole contents (% of remaining lansoprazole relative the lansoprazole weighed in) in various lansoprazole-stabilizer mixtures after the stress test. As described above, the humid environment (HE) is created by placing a glass insert containing 20 μl of DI water in the chromatography vial wherein the relative humidity is a function of the stress temperature selected. Vials without the glass insert are termed “non-humid environment (NHE).”

TABLE 1
Stability of lansoprazole in various drug/stabilizer mixture

| | | Remaining drug |
|---|---|---|
| Study | | (% of pre- |
| No. | Composition* | stress amount) |
| 1 | Lan, NHE | 99.25 |
| | Lan, HE | 0.88 |
| 2 | Lan + cholestyramine-Cl, NHE | 99.66 |
| | Lan + cholestyramine-Cl, HE | 0.94 |
| 3 | Lan + cholestyramine-OH, NHE | 83.63 |
| | Lan + cholestyramine-OH, HE | 65.64 |
| 4 | Lan + Na₂CO₃, NHE | 101.13 |
| | Lan + Na₂CO₃, HE | 95.85 |
| 5 | Lan + Eudragit E PO, NHE | Not done |
| | Lan + Eudragit E PO, HE | 95.98 |

*Lan: Lansoprazole;
NHE: non-humid environment;
HE: humid environment.

TABLE 2
Stability of lansoprazole in lansoprazole-chitosan mixture

| Study No. | Condition* | Wt (mg) | Peak area | % remaining | Average |
|---|---|---|---|---|---|
| | Standard | 29.55 | 1227081 | | |
| 1 | 200%, heat, NHE | 30.23 | 1255739 | 100.02 | |
| | | 30.23 | 1249232 | 99.50 | 99.76 |
| | 200%, heat, HE | 32.15 | 1318013 | 98.71 | |
| | | 32.15 | 1320614 | 98.91 | 98.81 |
| 2 | 100%, heat, NHE | 32.64 | 1354361 | 99.91 | |
| | | 32.64 | 1355332 | 99.98 | 99.95 |
| | 100%, heat, HE | 31.95 | 1325528 | 99.89 | |
| | | 31.95 | 1335832 | 100.67 | 100.28 |
| 3 | 75%, heat, NHE | 31.80 | 1338494 | 101.35 | |
| | | 31.80 | 1335213 | 101.1 | 101.22 |
| | 75%, heat, HE | 32.79 | 1341713 | 98.52 | |
| | | 32.79 | 1344930 | 98.76 | 98.64 |
| 4 | 50%, heat, NHE | 29.74 | 1250040 | 101.21 | |
| | | 29.74 | 1243110 | 100.65 | 100.93 |
| | 50%, heat, HE | 31.25 | 1272830 | 98.07 | |
| | | 31.25 | 1281548 | 98.74 | 98.41 |
| 5 | 25% heat, NHE | 31.09 | 1297958 | 100.52 | |
| | | 31.09 | 1292938 | 100.13 | 100.33 |
| | 25%, heat, HE | 27.85 | 1091567 | 94.37 | |
| | | 27.85 | 1097701 | 94.90 | 94.64 |
| 6 | 10%, heat, NHE | 29.41 | 1219390 | 99.83 | |
| | | 29.41 | 1226740 | 100.43 | 100.13 |
| | 10%, heat, HE | 32.58 | 1337614 | 98.86 | |
| | | 32.58 | 1327713 | 98.12 | 98.49 |

*Percent (%) is defined as relative to the ratio of drug substance weight to stabilizer weight (e.g., 1:1 = 100%; 1:2 = 200%). Heat: 60° C. for 10 days.
NHE: non-humid environment;
HE: humid environment.

The results in Tables 1 and 2 demonstrate that lansoprazole is stable in a dry environment but is unstable under humid conditions. As expected from published patents, sodium bicarbonate provide good stabilization effect for lansoprazole in the stress test. Cholestyramine chloride, however, did not provide much stabilization for the drug substance under humid conditions. In contrast, cholestyramine hydroxide unexpectedly provides significant stabilization effect toward lansoprazole in humid conditions. The degradation of lansoprazole in the lansoprazole- cholestyramine-OH mixture that was observed under dry conditions is likely to be caused by incomplete drying of the cholestyramine-OH resin after the counter ion conversion. As shown in Tables 1 and 2, Eudragit E-PO and chitosan also unexpectedly provide excellent stabilizing effect for lansoprazole. These data suggest that polymeric bases such as cholestyramine-OH, Eudragit E-PO, and chitosan can be used as stabilizers for benzimidazole derivative proton pump inhibitors, such as lansoprazole, pantoprazole, leminoprazole, pariprazole, omeprazole, rabeprazole, and esomeprazole, without disadvantages to gastroespophageal reflux patients. Moreover, the stabilizing composition can be easily manufactured by simply admixing the polymeric base with the benzimidazole derivative proton pump inhibitors.

### Example 4

### HPLC Method for Detecting Lansoprazole

The HPLC conditions for detecting lansoprazole is listed below:

| Phosphate | Adding 2.76 g of Sodium phosphate monobasic in 1200 | |
|---|---|---|
| buffer: | ml of HPLC water | |
| Mobile | Acetonitrile/phosphate buffer (v/v, 40/60), filter, | |
| phase: | and degas. | |
| Column: | 4.6 mm × 150 mm Supelcosil LC-8 DB column, 5 μm | |
| | in diameter. | |
| Injection | 10 μl | |
| volume: | | |
| Flow Rate: | 1.0 ml/min. | |
| Wavelength: | 285 nm | |
| Diluent: | 1% diethanolamine in water | |
| Retention | Intact lansoprazole: | single peak at 4.9 min. |
| time: | Degraded lansoprazole: | multiple peaks at 2.1-3.3 min. |

Having described preferred embodiments of the composition of the present invention and methods of making and using the same (which are intended to be illustrative and not limiting), it should be understood that modifications and variations can be made by persons skilled in the art in light of the above teachings. These modifications and variations are within the scope of what is described as defined by the appended claims.

What is claimed is:

1. A composition of matter comprising:
a benzimidazole derivative proton pump inhibitor in physical admixture with a polymeric base selected from the group consisting of cholestyramine-OH, Eudragit E-PO, chitosan, and a mixture of any two or more thereof,
wherein the polymeric base is present in sufficient amounts to retard degradation of the benzimidazole derivative proton pump inhibitor.

2. The composition of claim 1, wherein the benzimidazole derivative proton pump inhibitor is selected from at least one of the group consisting of lansoprazole, pantoprazole, leminoprazole, pariprazole, omeprazole, rabeprazole, and esomeprazole.

3. The composition of claim 2, wherein the benzimidazole derivative proton pump inhibitor comprises lansoprazole.

4. The composition of claim 1, wherein the polymeric base comprises cholestyramine-OH.

5. The composition of claim 1, wherein the polymeric base comprises Eudragit E-PO.

6. The composition of claim 1, wherein the polymeric base comprises chitosan.

7. The composition of claim 1, wherein the benzimidazole derivative proton pump inhibitor and the polymeric base are both in powder form.

8. The composition of claim 7, wherein the benzimidazole derivative proton pump inhibitor to polymeric base weight ratio is between 0.1 and 0.9.

9. The composition of claim 8, wherein the benzimidazole derivative proton pump inhibitor to polymeric base weight ratio is between 0.2 and 0.8.

10. The composition of claim 1, wherein said benzimidazole derivative proton pump inhibitor and said polymeric base are of pharmaceutical grade, said composition further comprising at least one pharmaceutically acceptable excipient.

11. The composition of claim 10, wherein the pharmaceutically acceptable excipient is one of a surfactant, plasticizer, filler, lubricant, preservative, sweetener agent, flavoring agent, pharmaceutical-grade dye or pigment, and viscosity agent.

12. The composition of claim 1, formulated in a dosage form for oral administration.

13. The composition of claim 12, further comprising an enteric coating.

14. A method for stabilizing a benzimidazole derivative proton pump inhibitor in a composition, said method comprising the step of:
admixing the benzimidazole derivative proton pump inhibitor with an amount of polymeric base selected from the group consisting of cholestyramine-OH, Eudragit E-PO, chitosan, and mixtures thereof sufficient to stabilize said benzimidazole derivatiave proton pump inhibitor against degradation.

15. The method of claim 14, wherein the benzimidazole derivative proton pump inhibitor is selected from the group consisting of lansoprazole, pantoprazole, leminoprazole, pariprazole, omeprazole, rabeprazole, and esomeprazole.

16. The method of claim 14, wherein the benzimidazole derivative proton pump inhibitor comprises lansoprazole.

17. The method of claim 14, wherein the polymeric base comprises cholestyramine-OH.

18. The method of claim 14, wherein the polymeric base comprises Eudragit E-PO.

19. The method of claim 14, wherein the polymeric base comprises chitosan.

20. The method of claim 14, wherein the benzimidazole derivative proton pump inhibitor and the polymeric base are both in powder form.

21. The method of claim 20, wherein the benzimidazole derivative proton pump inhibitor is mixed with the polymeric base at a benzimidazole derivative/polymeric base weight ratio of between 0.1 and 0.9.

22. The method of claim 21, wherein the benzimidazole derivative proton pump inhibitor is mixed with the polymeric base at a benzimidazole derivative/polymeric base weight ratio of between 0.2 and 0.8.

23. A method for prophylaxis or treatment of gastric acid disorders, comprising the step of administering to a patient in need of such treatment a therapeutically effective amount of the composition of claim 1.
